(19) United States
(12) Patent Application Publication
(10) Pub. No.: US 2009/0076428 A1
(43) Pub. Date: Mar. 19, 2009

(54) Ambidextrous ankle support

(76) Inventor: Jason Kay, Morristown, NJ (US)

Correspondence Address: Gearhart Law, LLC, 4 Ferndale Rd., Chatham, NJ, 07928, US

(21) Appl. No.: 12/221,649

(22) Filed: Aug. 5, 2008

Related U.S. Application Data
(60) Provisional application No. 60/964,148, filed on Aug. 9, 2007.

(51) Int. Cl. A61F 5/00

(52) U.S. Cl. 602/27; 602/36

(57) ABSTRACT

An ankle brace, comprising a truss and an envelope, the envelope having left and right wrapping straps and a truss sleeve suitable for securably holding said truss, and the truss having a bottom connected to left and right uprights having left and right ends, wherein said left and right uprights have equally sized ankle protrusions. The ankle brace is preferably constructed from nylon coated neoprene. The left and right straps of the invention allow a user to adjust the tension of the brace around the user's ankle. The equally sized ankle protrusions on the truss allow the brace to be worn on either the left or the right foot.

### CLAIM OF PRIORITY

This application claims the priority of Provisional Application U.S. Ser. No. 60/964148 filed date Aug. 9, 2007, which is incorporated herein by reference.

### FIELD OF THE INVENTION

The invention relates to medical devices, in particular, ankle braces for supporting weakened or injured ankles.

### BACKGROUND OF THE INVENTION

It is estimated that between 23,000 and 27,000 ankle sprains happen each day in the United States, making ankle injuries one of the most common injuries. Ankle injuries occur most often in athletes participating in sports with side to side movement, such as basketball, tennis and racquetball, although just about all sports create their fair share of ankle sprains. Since weakened ligaments are more easily re-injured, the re-injury rate for ankle sprains is high and can approach 70% in sports such as basketball.

Anatomically, the ankle includes the talus or ankle bone and the ankle mortise created by the lower tibia (inner ankle bone, leg bone) and fibula (outer ankle, leg bone). There are three main outer ankle ligaments. The anterior talo-fibular ligament, the calcaneo-fibular ligament and the posterior talo-fibular ligament. Other structures in this area include the peroneal tendons (which can sublux or move out of place), the calcaneo-cuboid ligament, and the base of the 5th metatarsal (which can break).

The present invention relates to medical devices, and in particular, a support brace that can be used either on the left or right foot. The brace can provide support for a weakened or injured ankle. Depending on the severity of the injury this brace may be used by an athlete with ankle problems, providing the support needed for the athlete to continue training or competing. In the case of more severe injuries, the ankle brace of the present invention can provide medium levels of support, while the ankle heals. The present invention is advantageous in that the brace is ambidextrous and can be worn either on the left or the right foot of the user. Offering an ambidextrous brace offers obvious advantages to distributors and retailers who would not need to stock as much inventory, and to the user as well, who needs to purchase only one brace for injuries to either the left or the right ankle. In addition, the brace of the present invention offers a portion of a truss that is parallel to the wearer's leg providing the wrapping straps disclosed herein more surface area for attachment, thus improving the support offered by the user's brace.

It is an object of the invention to provide a brace that can support a user's ankle.

It is another object of the invention to provide an ambidextrous brace that can be worn on an ankle.

It is also an object of the invention to provide a brace having two wrapping straps.

It is an object of the invention to provide a brace that has a truss, a portion of which runs in parallel to a user's leg.

It is an object of the invention to teach a brace that can be worn while the user is wearing a shoe or other footwear.

It is an object of the invention to provide a brace with two wrapping straps that allow the user to adjust the tension of the brace around the user's ankle.

It is another object of the invention to provide a brace having reinforced loop assemblies.

It is also an object of the invention to provide an ankle brace that can be used in lieu of taping a person's ankle.

It is further an object of the invention to provide an ankle brace that can be used to prevent injury or to prevent re-injury to a person's ankle.

### SUMMARY OF THE INVENTION

The present invention is an ankle brace, comprising a truss and an envelope, the envelope having left and right wrapping straps and a truss sleeve suitable for securably holding said truss, and the truss having a bottom connected to left and right uprights having left and right ends, wherein said left and right uprights have equally sized ankle protrusions. The ankle brace is preferably constructed from a breathable and stretchable fabric such as nylon-coated neoprene. The left and right straps of the invention allow a user to adjust the tension of the brace around the user's ankle. The equally sized ankle protrusions on the truss allow the brace to be worn on either the left or the right foot.

### BRIEF DESCRIPTION OF THE DRAWINGS

FIG. 1 is a top view of the preferred embodiment of the invention when it is unassembled.

FIG. 2 is a front view of a preferred embodiment of the invention after it has been assembled.

FIG. 3 is a right side view of a preferred embodiment of the invention, showing the invention being placed on a user's right foot.

FIG. 4 it is a right side view of a preferred embodiment of the invention, showing the orthopedic device completely secured on the user's foot.

FIG. 5 is a left side view of a preferred embodiment of the invention, showing the invention being placed on a user's right foot.

FIG. 6 is a left side view of a preferred embodiment of the invention, showing the orthopedic device completely secured on the user's foot.

FIG. 7 is a front view of a preferred embodiment of the invention, showing the orthopedic device completely secured on the user's left foot.

FIG. 8 is a perspective view of a truss used in a preferred embodiment of the present invention.

FIG. 9 is a perspective view of a truss used in an alternate embodiment of the same present invention.

FIG. 10 is a right side view of an alternative embodiment of the present invention, showing the orthopedic device completely secured on the user's foot.

### DESCRIPTION OF THE PREFERRED EMBODIMENTS

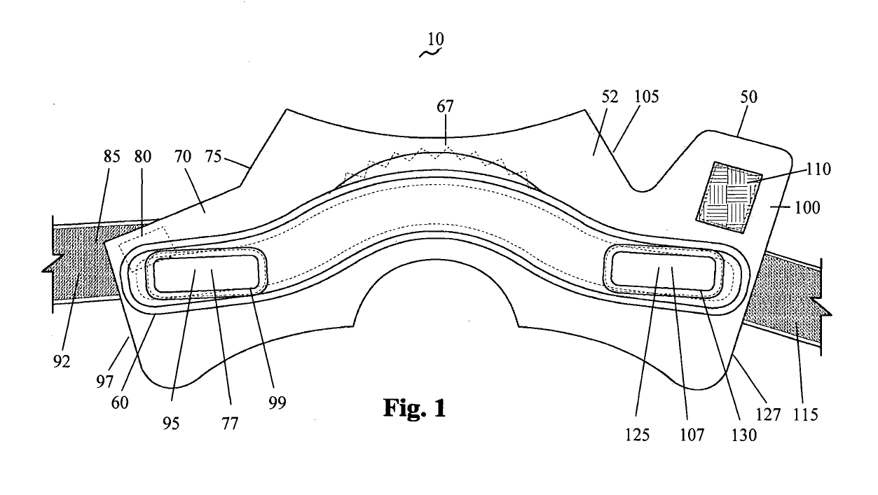

The preferred embodiments of the present invention will now be described with reference to FIGS. 1-8 of the drawings. Identical elements in the various figures are identified with the same reference numerals.

Starting now with the figures, FIG. 1 shows the invention 10, having envelope 50, internal surface 52, truss sleeve 55, truss sleeve reinforcement 60, truss access 67, left side 70, left seam 75, left side fastener 80, left wrapping strap 85, left wrapping strap fastener 92, left strap loop 95, rear left seam 97, and left loop reinforcement 99. Also seen is right side 100, right seam 105, right side fastener 110, right wrapping strap 115, right strap loop 125, rear right seam 127, and right loop reinforcement 130.

A preferred material for envelope 50 is polypropylene coated neoprene. Other materials for envelope 50 include cotton, nylon, leather, canvas, foam, plastic, spandex rubber, or any other suitable fabric, preferably a material that has at lease some elastic and/or breathability properties. Other suitable materials could be combinations of any of the above-mentioned materials.

Truss access 67 should be noted in reference to FIG. 1. Truss access 67 allows the U shaped truss 200 to be inserted into truss sleeve 55. Thus, the opening defined by truss access 67 allows left upright 225 and right upright 240 be slid into truss sleeve 55.

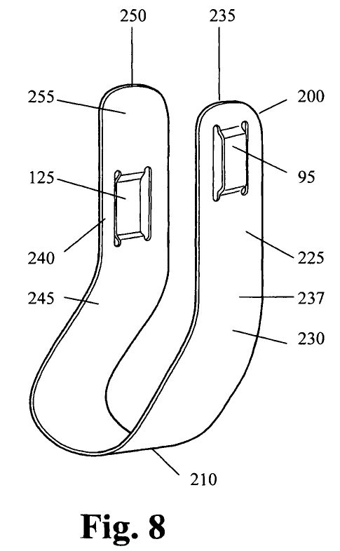

In a highly preferred embodiment, the invention has left strap loop 95, and right strap loop 125. Preferably, these left and right strap loops are disposed on truss 200, as seen in FIG. 8. These left and right strap loops are adapted to receive left wrapping strap and 85 and right wrapping strap 115. Envelope 50 has corresponding left loop slit 77 and right loop slit 107, so that when truss 200 is inserted into trust leave 55 left strap loop 95 and right strap loop 125 protrude so left wrapping strap 85 and right wrapping strap 115 can be inserted through left and right strap loops 95 and 125 while the braces are being put on as described below. Note that left strap loop 95 and right strap loop 125 may be placed anywhere along left upright 225 or right upright 240. In a preferred embodiment, one of the left or right strap loops, 95 and 125, respectively, may be different distances from left and right upright ends 235 and 250, as illustrated in FIG. 8.

In an alternative embodiment, loop reinforcement 99, and right loop reinforcement 130 can be attached to envelope 50. These left and right reinforcements are adapted to receive specially shaped loop assemblies that enable loops 125 and 95 to be rigorously connected to envelope 50. In this embodiment, the loop assembly has a flat shaped rectangular piece made out of engineered resins such as ABS, polycarbonate and other blends or composites. The rectangular piece has the loop and hook formed into it, or alternatively, the hook is attached to the plastic. The loop reinforcing piece is slid through a hole in envelope 50 at left loop slit 77 and right loop slit 107. It is then sewn over back of rectangular piece loop 125, thereby securably holding the rectangular piece in position. As seen in FIG. 1, left loop reinforcement 99 and right loop reinforcement 130 may be double stitched to ensure that rectangular piece is held in place securely.

Right side fastener 110 and left side fastener 80 can be any type of fastener suitable for use in the present invention. It is most likely that left and right fasteners 110 and 80 are of the hook and loop type, commonly known as Velcro®. While hook and loop fasteners are the preferred types of fasteners it is envisioned that other types of fasteners could be suitable as well including buckles, laces, snaps, hook and eye closures, buttons, adhesives, as well as straps relying on friction.

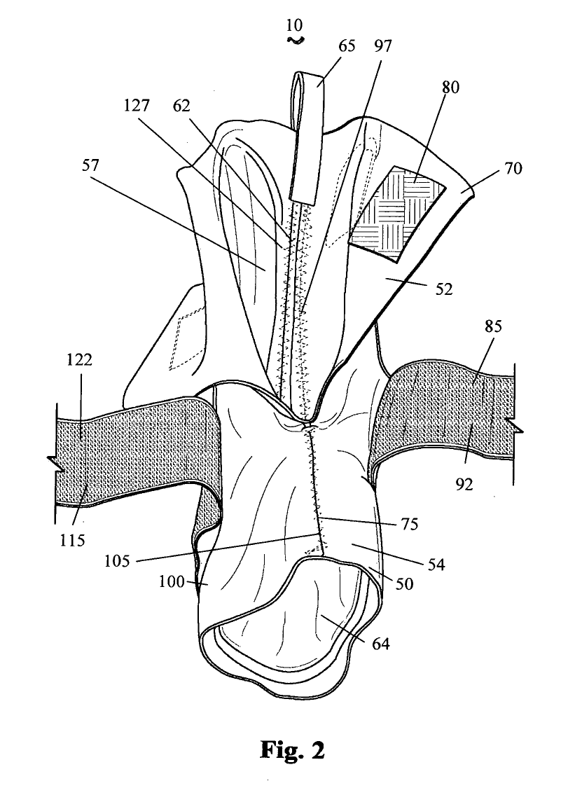

Truss sleeve 55 is attached to external surface 54 (FIG. 2) of envelope 50. Truss sleeve 55 can be attached to envelope 50 in any manner, so long as truss sleeve 55 is securely attached to truss envelope 50. FIG. 1 shows truss sleeve 55 being attached to external surface 54 (FIG. 2) by means of sewing truss sleeve 55 onto envelope 50, since it is important to have a strong connection between truss sleeve 55 and envelope 50. It may be desirable to double stitch truss sleeve 55 on envelope 50. Other methods of attachment, however, are also envisioned depending on the materials used, and could include adhesive attachment, integral molding, attachment using hook and loop fasteners, or any other suitable means.

The size of the shape of truss sleeve 55 should be configured and adapted to the shape of truss 200, and should be sized so that there is a minimum of movement of the envelope 50 around truss 200. In most cases this will mean that truss 200 fits snugly into truss sleeve 55.

The size of envelope 50 will of course depend on the size of the user's foot. It is expected that the size of the truss envelope and the shape can be varied to accommodate different feet of different users. For example, different sizes of envelope 50 could be created to fit users having small, medium and large sized feet. Sizes could also vary depending on the age or the gender of the user. Dimensions of envelope 50 can range from 21″×9″ but can range from 30″×15″ down to 10″ by 4″. The thickness of the envelope 50 is ideally about 1/16″ by can rage from 1/64″ to ¼″.

FIG. 2 shows envelope 50, internal surface 52, external surface 54, truss reinforcement 62, loop 65, left side 70, left seam 75, left side fastener 80, left wrapping strap 85, left wrapping strap fastener 92, rear left seam 97, and right side 100 and right seam 105, right wrapping strap 115, right wrapping strap fastener 122, and rear right seam 127. In addition, FIG. 2 shows top opening 57 and front opening 64.

Truss reinforcement 62 is an optional reinforcement strap that reinforces the seam where the left side 70 and right side 100 are joined together at rear left seam 97 and rear right seam 127. It can be made of any of the materials suitable for envelope 50 described above. In addition, attached to reinforcement 62 is loop 65. Loop 65 can also be made from materials similar to truss reinforcement 62. Loop 65 can help the user pull envelope 50 over the user's foot.

One unique feature of the invention is that a single brace can be used to accommodate either the right or the left foot of the user. This is in contrast to other braces in the prior art, which are specific to either the left foot or the right foot. The ambidextrous nature of the brace taught in the present invention allows the user to use the brace for either ankle if so desired. Also, it is of greater convenience to the distributor or the retailer or the physician since it allows them to reduce their inventory by half. It also reduces the possibility of the user receiving the incorrect product by narrowing the choices to size alone. In addition, production costs are lower because higher volumes of a single product can be manufactured instead of manufacturing smaller volumes of two individual products, thus lowering the cost to the user.

Also seen in FIG. 2 are left seam 75, and right seam 105, and rear left seam 97 and rear right seam 127. As seen in the drawings the seams are connected together using ordinary stitching, however other means of fastening the respective left and right seams together can be accomplished in any number of ways. For example, the edges could be thermally sealed together, or the seams could be connected using zippers or adhesives.

In FIG. 1, left strap loop 95 and right strap loop 125 are configured and secured to allow left and right wrapping straps 85 and 115 (FIG. 1 and FIG. 2) to be circulated around the user's ankle. The loops must be of sufficient strength to withstand the stress created by tightening left wrapping strap 85 and right wrapping strap 115, and strong enough to withstand the substantial physical forces that brace will be subjected to. The loops may be integral with truss 200 as seen in FIG. 8. The loops can be made from any suitable materials such as metal, plastic, rubber cloth, ABS, etc., with the most preferred material being ABS or other plastic, or metal.

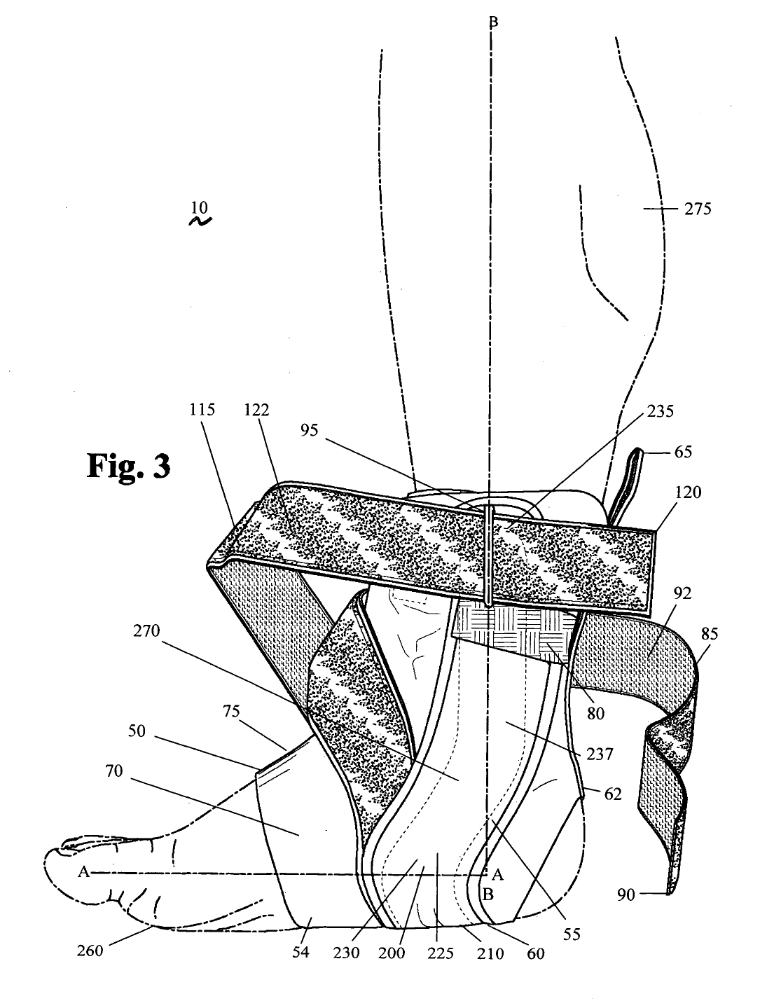

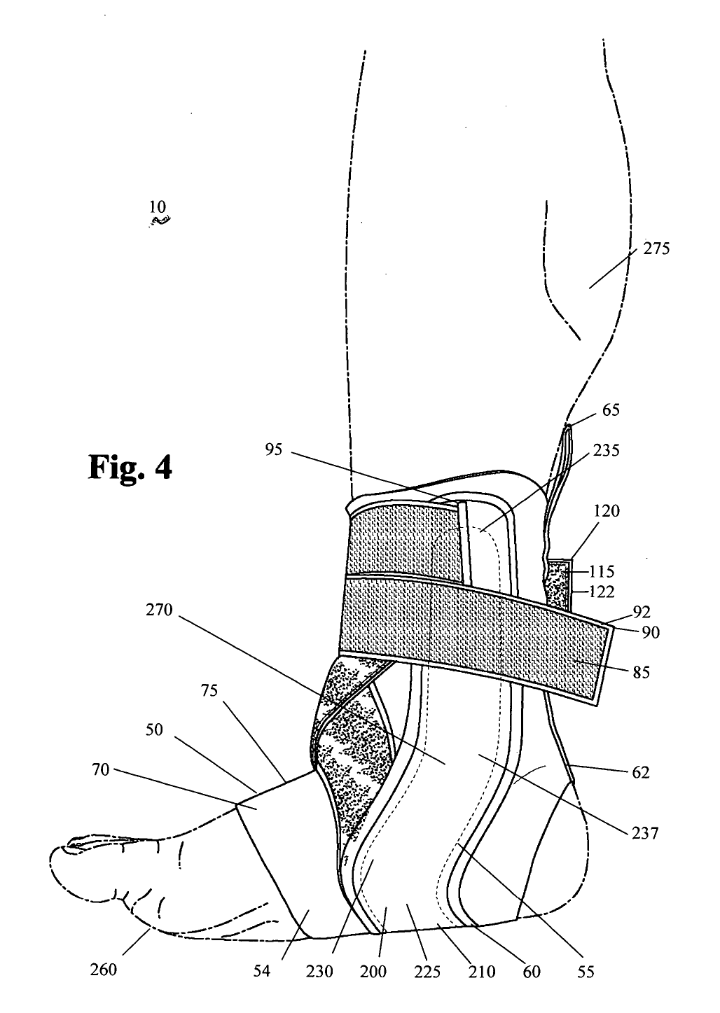

As seen in FIGS. 3 and 4, invention 10 has envelope 50, external surface 54, truss sleeve 55, truss sleeve reinforcement 60, truss reinforcement 62, loop 65, left side 70, left seam 75, left side fastener 80, left wrapping strap 85, left wrapping strap end 90, right wrapping strap fastener 92, left strap loop 95, right wrapping strap 115, right wrapping strap end 120, right wrapping strap fastener 122, truss 200, bottom 210, left upright 225, left upright protrusion 230, left upright end 235, left perpendicular 237, human foot 260, and calf 275.

As can be seen in FIG. 3, lines AA and BB define a right angle where line AA is parallel to the plane defined by the user's foot and line BB is parallel as defined by the user's leg. The intersection of the lines AA and BB is approximately at the center of the mellealus and talus. The brace angles forward to clear the calcaneus and allow the foot to pivot in plantarflexion and dorsiflexion, but restrict pronation and supination. Line AA and line BB form roughly a right angle. Line BB and left perpendicular 237 are also parallel. Thus, there is a substantial portion of truss 200 that is perpendicular to the plane defined by line AA and parallel to line BB.

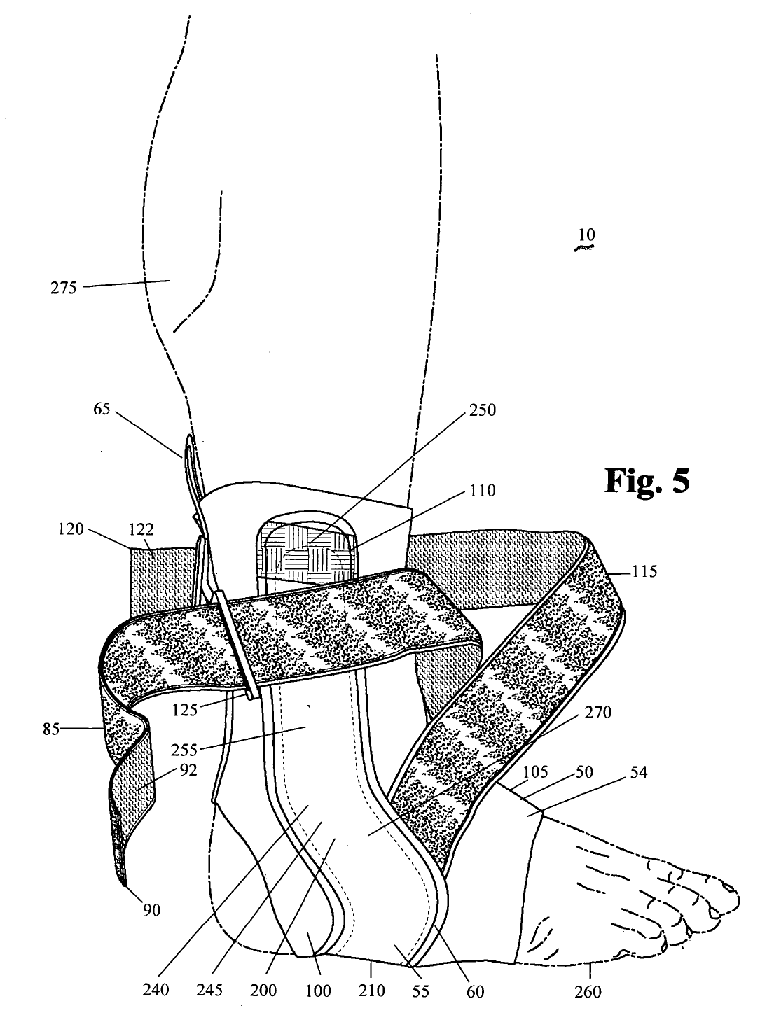

Having portions of truss 200 parallel to the user's leg significantly improves the stability of the brace. Because a significant portion of the truss is parallel to the user's leg, left wrapping strap 85 and right wrapping strap 115 have more area around the truss 200 to grip around the user's leg. The additional surface area offered by left perpendicular 237 and right perpendicular 255 on left upright 235 and right upright 240 (FIG. 5) results in additional stability to the user's ankle, that is not offered in prior art ankle braces where the truss is at an angle orientated in relation to the user's leg or ankle.

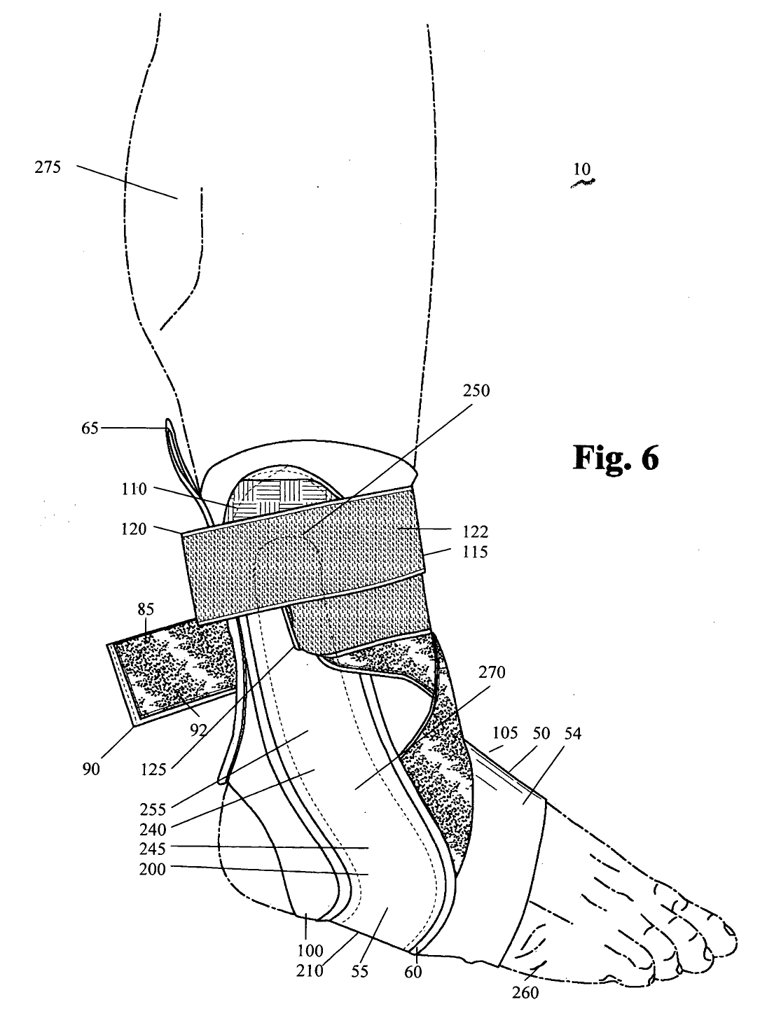

The truss is formed so lengths of left perpendicular 237 (FIG. 3) and right perpendicular 255 (FIG. 6) are parallel to the user's leg. As used herein, the terms “left perpendicular 237” and “right perpendicular 253” are defined as the portion of the left upright protrusion 230 and right upright protrusion 245 (FIG. 5) which are effectively parallel to line BB as shown in FIG. 3. In different embodiments of the invention, anywhere from 10 to 50% of the total length of left upright 225 and right upright 240 can be substantially perpendicular to the user's leg. In a particularly preferred embodiment, 30% of the length of said left upright 225 and 30% of the length of said right upright 240 are substantially perpendicular to a wearer's leg, i.e., the length of left perpendicular 237 is 30% of the length of left upright 225 and the length of right perpendicular 255 is 30% of the length of right upright 240.

In FIGS. 5 and 6, invention 10 has envelope 50, external surface 54, truss sleeve 55, truss sleeve reinforcement 60, loop 65, left wrapping strap 85, left wrapping strap end 90 and left wrapping strap fastener 92. In addition, invention 10 has right side 100, right seam 105, right side fastener 110, right wrapping strap 115, right wrapping strap end 120, right wrapping strap 122, right strap loop 125, truss 200, bottom 210, right upright 240, right upright protrusion 245, right upright end 250, right perpendicular 255 human foot 260, and calf 275.

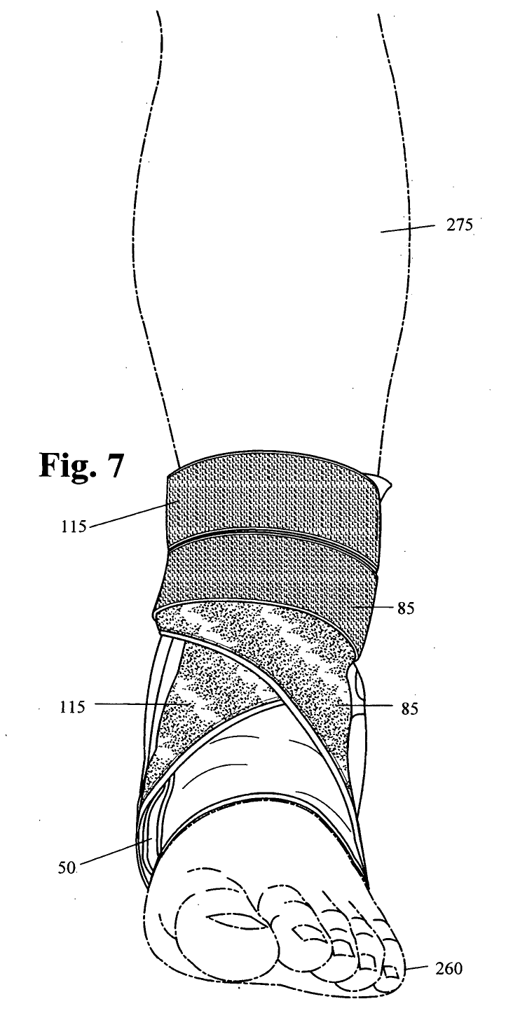

FIG. 7 shows envelope 50, right wrapping strap 115, and left wrapping strap 85, foot 260, and calf 275. FIG. 7 illustrates a front view of the brace after it is completely put on the user's ankle.

FIGS. 3 and 4 as well as FIGS. 5 and 6 illustrate how the brace looks as it is being put on by the wearer. In order to put the brace on, the user inserts their foot through the top opening 57 (FIG. 2) of the brace, their foot and toes extended through front opening 64 (see FIG. 2). Then, right wrapping strap end 120 is slid through left strap loop 95. Next, the user inserts left wrapping strap end 90 through right strap loop 125. The user then pulls left wrapping strap end 90 and right wrapping strap end 120 to exert enough force to create a tight fit of envelope 50 around user's ankle 270. The user continues to wrap right wrapping strap 115 and left wrapping strap 85 until the user's ankle is completely wrapped as seen in FIG. 7.

Notably, the present invention is further distinguished from some braces in the prior art because it utilizes left and right wrapping straps 85 and 115 rather than a single wrapping strap. The left and right wrapping straps allow the user more flexibility for tightening the brace around the user's ankle. Also, by locking the tops of the stays together, and using the shoe to lock the bottoms together, the brace can be thinner and still give the support needed by keeping the foot from rotating inside the brace. The straps keep the stays bound tight and thus allowing them to support or exert a force on the leg relative to the foot to prevent the supination.

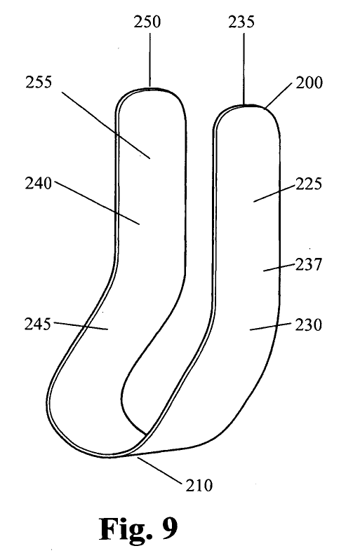

FIGS. 8 and 9 are a view of the truss 200. FIG. 8 shows a preferred embodiment of the truss having loops 95 and 125 disposed thereon, whereas FIG. 9 shows an embodiment without loops 95 and 125. In the alternate embodiment the truss 200 would not have reinforcing loops 95 and 125. In both figures, truss 200 has bottom 210, left upright 225, left upright protrusion 230, left upright end 235, left perpendicular 237, right upright 240, right upright protrusion 245, right upright end 250, right perpendicular 255. The truss 200 of the present invention can be constructed of any suitable material, such as metal, (especially spring grade metals) plastic, wood, or composite materials. Particularly preferred materials for the truss are molded materials that have some degree of flexibility, such as nylon or poly propylene. Other preferred materials would include ABS, ASA, PC, PPO, PPE, PS, PBT, PET, Polyester (PE),PEI, PVC or some blend of one or more of the above resins.

Importantly, the truss is configured so it has a relatively homogenous shape. For example, protrusions 245 and 230 are equally shaped as seen in FIG. 8. This is distinguished from other braces where the shape of the truss protrusions are different depending on whether the brace is intended to support the left or right ankle of a wearer. This allows for reduced inventory costs for the retailer or distributor of the brace, and in addition, allows the user to wear the brace on either the left or the right foot as occasion demands. The preferred truss overall length is 18″, but can range from 8″ to 24″. The preferred thickness of the truss is ⅛″ but can range from 1/64″to 1″. The preferred width of the truss is 1¼″ but can range from ½″ to 3″. It is preferred that the truss is constructed so that the right and left upright protrusions are substantially equal in length, but in some cases they may be made of differing lengths.

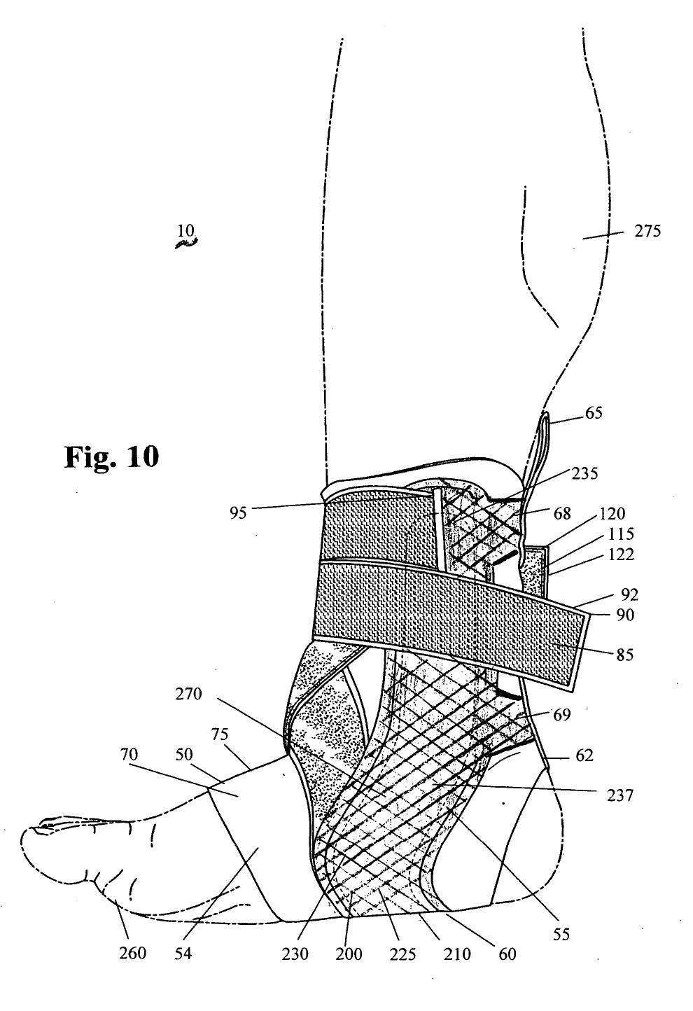

FIG. 10 shows an alternate embodiment of the invention 10. Shown in FIG. 10 is lower sleeve reinforcement 68 and upper sleeve reinforcement 69. The sleeve reinforcements wrap around the back of the ankle to join the back left and right sections of the truss. The purpose of the sleeve reinforcements is to provide extra stability to the truss by preventing the truss from shifting during movement.

Although two sleeve reinforcements are preferred as illustrated in FIG. 10, there may be any number of sleeve reinforcements, although preferably from one to five, and they may extend for any fraction of the truss 200, from none of the truss to the entire length of the truss, or any portion thereof. The sleeve reinforcements may be used with or without the left wrapping strap 85 and right wrapping strap 115. The sleeve reinforcements may be an integral part of the truss sleeve 55 or they may be detachable. The sleeve reinforcements may wrap around the back of the ankle as shown in FIG. 10, or they may wrap around the front of the ankle, around any portion of the ankle, or around the entire ankle.

The sleeve reinforcements may be constructed from any suitable material, including but not limited to, polypropylene coated neoprene, cotton, nylon, leather, canvas, foam, plastic, spandex rubber, elastane, or any other suitable fabric. Other suitable materials could be combinations of any of the above-mentioned materials or any of these materials with other materials.

FIG. 10 also shows envelope 50, external surface 54, truss sleeve 55, truss sleeve reinforcement 60, truss reinforcement 62, loop 65, left side 70, left seam 75, left wrapping strap 85, left wrapping strap end 90, right wrapping strap fastener 92, left strap loop 95, right wrapping strap 115, right wrapping strap end 120, right wrapping strap fastener 122, truss 200, bottom 210, left upright 225, left upright protrusion 230, left upright end 235, left perpendicular 237, human foot 260, ankle 270, and calf 275.

The brace of the present invention can also be worn while the user is engaged in athletic activity and is designed to fit so that it can be worn while also wearing an athletic shoe or other footwear. The brace allows plantarflexion (point toes down) and dorsiflextion (point toes up) but resists pronation (roll foot outwards) and supination (roll foot inwards). The brace can be worn with dress shoes, athletic shoes, boots (hiking, work, dress, etc.) athletic shoes, walking shoes or any shoe that applies at least some minimal force around the ankle.

Moreover, the nature of the wrapping straps and the fasteners allows the brace to easily be adjusted during the athletic activity or use, and can be tightened or loosened as desired by the user offering different degrees of support and comfort, depending on the needs and the desires of the user.

It is also important to note that while the present invention can be used to support an injured ankle during athletic activity, it can also be used to prevent injury. For example, it is common in athletic competitions to tape the athlete's ankles prior to the athletic activity to reduce the possibility of injury. The process of taping is cumbersome and uncomfortable for the athlete, and it is of course painful to remove the tape. The present invention can be used in lieu of a taping, having the advantage that it is not difficult or painful to remove, and it can also be removed and put back on at will at any time during the competition. The athlete does not have to go through the process of removing the tape, taping the ankle again, then removing the tape a second or third time. Moreover, the same is true in the case of ankles that have been injured and have healed. The present invention reduces the likelihood of a re-injury. It is expected that many devices of the present invention will be sold and marketed for preventative or prophylactic purposes, i.e. to prevent injury or re-injury.

Although this invention has been described with a certain degree of particularity, it is to be understood that the present disclosure has been made only by way of illustration and that numerous changes in the details of construction and arrangement of parts may be resorted to without departing from the spirit and the scope of the invention.

I claim:

1. An ankle brace, comprising:
a truss and an envelope,
the envelope having left and right wrapping straps and a truss sleeve suitable for securably holding said truss; and
the truss having a bottom connected to left and right uprights having left and right ends,
wherein said left and right uprights have equally sized ankle protrusions.

2. The ankle brace of claim 1, wherein said uprights are fashioned in a manner so at least 10 percent of the length of said ends are substantially perpendicular to a wearer's leg.

3. The ankle brace of claim 1, wherein said uprights are fashioned in a manner so at least 30 percent of the length of said ends are substantially perpendicular to a wearer's leg.

4. The ankle brace of claim 1, wherein said uprights are fashioned in a manner so at least 50 percent of the length of said ends are substantially perpendicular to a wearer's leg.

5. The ankle brace of claim 1, wherein said envelope has a left front portion and a right front portion, and said left and right front portions may be fastened together.

6. The ankle brace of claim 1, wherein at least one wrapping strap may be attached to the left or right truss.

7. The ankle brace of claim 1, wherein the wrapping straps are attached to the envelope with a hook and loop fastening system.

8. The ankle brace of claim 1, wherein the brace has at least one truss reinforcement.

9. The ankle brace of claim 1, wherein the truss is made from plastic or metal.

10. The ankle brace of claim 1, wherein the truss is made of ABS.

11. The ankle brace of claim 1, wherein the truss is removable from the envelope.

12. An ambidextrous ankle brace, comprising:
a truss and an envelope,
the envelope having left and right wrapping straps and a truss sleeve suitable for securably holding said truss;
the truss having a bottom connected to left and right uprights having left and right ends,
wherein said uprights are fashioned in a manner so at least 10 percent of said ends are substantially perpendicular to the wearers leg; and
wherein said left and right uprights have ambidextrously fashioned ankle protrusions.

13. The ankle brace of claim 12, wherein said uprights are fashioned in a manner so at least 30 percent of the length of said ends are substantially perpendicular to a wearer's leg.

14. The ankle brace of claim 12, wherein said uprights are fashioned in a manner so at least 50 percent of the length of said ends are substantially perpendicular to a wearer's leg.

15. The ankle brace of claim 12, wherein said envelope has a left front portion and a right front portion, and said left and right front portions may be fastened together.

16. The ankle brace of claim 12, wherein at least one wrapping strap may be attached to the left or right truss.

17. The ankle brace of claim 12, wherein the wrapping straps are attached to the envelope with a hook and loop fastening system.

18. The ankle brace of claim 12, wherein the truss is made from plastic or metal.

19. The ankle brace of claim 12, wherein the truss is made of ABS.

20. The ankle brace of claim 12, wherein the brace has at least one truss reinforcement.

21. The ankle brace of claim 12, wherein the truss is removable from the envelope.

22. The ankle brace of claim 12, wherein the ankle brace is used as a preventive device to replace taping.

23. The ankle brace of claim 12, wherein the ankle brace is used to prevent injury or reinjury.
